(19) United States
(12) Patent Application Publication
(10) Pub. No.: US 2016/0113865 A1
(43) Pub. Date: Apr. 28, 2016

(54) HYDROGEN-GENERATING EFFERVESCENT TABLET AND METHODS THEREFOR

(71) Applicant: H2TAB LLC, Las Vegas, NV (US)

(72) Inventors: Mikhail Kazakevitch, Oceanside, CA (US); Tyler LeBaron, Rexburg, ID (US)

(73) Assignee: H2TAB LLC, Las Vegas, NV (US)

(21) Appl. No.: 14/922,767

(22) Filed: Oct. 26, 2015

Related U.S. Application Data
(60) Provisional application No. 62/069,619, filed on Oct. 28, 2014.

(51) Int. Cl. A61K 9/46; A61K 9/00; A23L 2/40; A61K 33/00

(52) U.S. Cl. CPC A61K 9/0007; A61K 33/00; A61K 9/0095; A23L 2/40; A23V 2002/00

(57) ABSTRACT

Convenient, inexpensive and portable effervescent tablets that the consumer can add to water to generate hydrogen-rich water just prior to drinking. The effervescent tablets include a base metal and an edible acid that, within about 5-10 minutes of mixing, generate a palatable aqueous solution having about 0.8 mM to about 3 mM hydrogen and a pH of 8-10.

### CROSS-REFERENCE TO RELATED APPLICATIONS

This application claims the benefit of U.S. Provisional Application No. 62/069,619 filed Oct. 28, 2014, which is incorporated herein by reference.

### BACKGROUND OF THE INVENTION

Using molecular hydrogen (H₂) as a nutritional supplement has found popularity in the United States and other countries. There are several well-known methods of generating molecular hydrogen for human consumption. For example, consumption of hydrogen-enriched or hydrogen-saturated water is a well-known delivery method. There are several known ways to produce such saturated hydrogen water using electrical and non-electrical devices. For example, electrical methods include water ionization or electrolysis. In another example, non-electrical methods include a variety of base metals trapped in the mechanical device. It is known that these methods produce a hydrogen concentration that is lower than the theoretical maximum, which is 0.8 mM hydrogen (H₂) at 1-atmosphere (atm) partial pressure of 100% hydrogen gas. Since these methods require bulky equipment, they are not portable and tend to be expensive. As a result, they are not considered to be consumer friendly. For years, researchers have searched for a method of producing a dry compound that can be added to potable water, to generate drinkable hydrogen-saturated water. Ideally, such a compound would be portable, cheap, safe and effective. This has proven very difficult to do, for the following reasons. Chemically generating hydrogen (H₂) in water requires mixing a base metal with an edible acid. Base metals are highly reactive and may even be flammable or explosive. Mixtures of base metals with organic acids are highly unstable. Because tablet manufacturing calls for extensive use of disintegrant and lubricant excipients, manufacturing such hydrogen-generating dry mixtures as tablets is not practical. In particular, these two types of excipients are hydrophobic non-water soluble chemicals. When mixed with water, these excipients produce a non-aesthetic unpalatable solution that the consumer perceives as undrinkable. Additionally, to facilitate production of hydrogen, organic acids are employed. The choice of organic acids must be made from anhydrous non-hygroscopic materials, such as malic acid, fumaric acid, and the like.

Therefore, there is a need for a dry compound that can be provided to the consumer as an effervescent tablet, powder, granule, or the like, that is mixable with water to generate drinkable hydrogen water that is aesthetically pleasing and palatable. Preferably, such a tablet, powder or granule would be provided in individual serving sizes, be convenient for use on-the-go, inexpensive, safe and effective.

### SUMMARY OF THE INVENTION

Drinking hydrogen water for medicinal and nutritional purposes is popular, but usually requires expensive, bulky equipment to make the hydrogen water, which has a very short shelf life due to volatile nature of hydrogen gas. The present invention is directed toward an inexpensive and conveniently portable effervescent tablet that can be dropped into water, such as a bottle or glass of water, to generate hydrogen water just prior to drinking. The effervescent tablet is dry and contains a base metal, such as metallic magnesium, and an edible acid. The metal reacts with water according to Formula I, below:

Me+H₂O->xH₂+Me_{y}(OH)ₓ I

With regard to Formula I, above, the term Me is used as abbreviation for the metal, x is the number of moles of the chemical species and y denotes the oxidation state of the metal Me. As is known in the art, the number of moles x of the chemical species depends upon the number of electrons donated from the metal Me. The acid will react with the OH⁻ species to form water (H₂O) and thus prevent the otherwise complexation of the Me(OH⁻)ₓ adduct, which results in passivation of the reaction. The end result is a tablet that rapidly reacts with water to generate molecular hydrogen (H₂), which dissolves into the water.

Other objects and advantages of this invention will become apparent from the following description taken in conjunction with any accompanying drawings wherein are set forth, by way of illustration and example, certain embodiments of this invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific chemical structure and functional details disclosed herein are not to be interpreted as limiting, but merely as a basis for the claims and as a representative basis for teaching one skilled in the art to variously employ the present invention in virtually any appropriately detailed chemical structure.

In a first embodiment, the present invention is directed toward a dry effervescent material or mixture that when added to water, generates one liter of drinkable solution containing molecular hydrogen (H₂) at a concentration of at least 0.8 mM, which may be referred to herein as simply hydrogen water. Once prepared, such as is described below, the user can consume the hydrogen water orally, such as by drinking. Alternatively, the hydrogen water can be applied topically to the skin and hair, such as is known in the art.

For convenient use, the effervescent mixture can be formed into a tablet, a powder or a granule. Tablets can be sized to provide individual serving sizes of the mixture, that is, the appropriate amount of mixture for a specific volume of water, such as one pint or one liter. Similarly, powders and granules can be aliquoted into single-serving-sized packets or cups. To facilitate the following discussion, the effervescent mixture will be referred to herein as an effervescent tablet that is intended to be a single-serving sized quantity of the effervescent mixture. It is understood that the effervescent mixture could be provided in larger or smaller quantities and in other forms known in the art.

An effervescent tablet of the present invention includes a base metal, denoted by Me in Formula I above, an edible non-hydroscopic dry organic acid, and an edible binding-excipient material. When the effervescent tablet is added to a quantity of potable water (H₂O), the base metal Me and the organic acid react to produce an aqueous solution with a molecular hydrogen (H₂) concentration of between 0.8 mM and 2.5 mM and a final pH of between 8 and 10 depending upon the quantity of water, such as depending upon the size of the bottle of water. Generally, the effervescent tablet is sized for mixing with between about 200 ml to about 2 liters of water while providing the required hydrogen concentration and pH. For example, since drinking water is often sold in 500 ml bottles, the effervescent tablet can be conveniently sized for use with such quantities of water.

As noted above, the effervescent tablet includes a base metal Me. Generally, the effervescent tablet includes at least 5 mg of the base metal Me and up to 200 mg of the base metal Me. For example, the effervescent tablet can be formulated to include between 30 mg and 60 mg of the base metal Me. The base metal Me used in the effervescent tablet is an active non-ionic metallic metal selected from the group consisting of alkaline earth minerals and other metals in their non-ionic metallic state. Preferred base metals Me have a greater negative redox potential than hydrogen gas (H₂), such as alkali metals and alkaline earth metals. Suitable base metals Me include, but are not limited to, strontium (Sr), calcium (Ca), magnesium (Mg), aluminum (Al), manganese (Mn), zinc (Zn), iron (Fe) and combinations thereof.

The effervescent tablet includes a quantity of an edible non-hydroscopic dry organic acid. Generally, when prepared for dilution into 200 ml to 2 liters of water, the effervescent tablet includes at least about 20 mg of the organic acid and up to about 300 mg of the organic acid. For example, in some circumstances, the effervescent tablet includes between 60 mg and 190 mg of the organic acid. However, the quantity of the organic acid is suitable for stoichiometric reaction with the quantity of the base metal Me provided in the tablet. Suitable edible dry organic acid include, but are not limited to, organic acids, such as carboxylic acids, which includes maleic acid, succinic acid, malic acid, fumaric acid, formic acid, oxalic acid, all stereoisomers or derivatives thereof, and all forms of alpha-keto acids, polycarboxylic acids and inorganic acids such as boric acids and certain Lewis acids. The amount of a selected edible dry organic acid used in a particular effervescent tablet depends upon the acid's dissociation constant (pKa) and the number of hydrogen ions (H+) that can be produced per acid molecule.

The stoichiometry of acids is such that they behave as acid/base buffers. When the pH of a solution (i.e., water) is acidic, the acid rapidly reacts with the base metal Me to quickly produce hydrogen gas (H₂). However, based on stoichiometry, the reaction will turn alkaline near its completion. The reaction kinetics are sufficient that in 5 minutes the pH is above the acid's pKa, resulting in a near H⁺ concentration of one base 10 log unit above the highest pKa of the organic acid, which is near a neutral pH and thus tasteless water. After 10-20 minutes the reaction has proceeded to sufficient completion so as to result in a super-saturated hydrogen concentration of between 1 mM and 3 mM. The edible dry organic acid of the effervescent tablet ranges from 100 mg to 500 mg, preferably 100 mg to 300 mg and may include individual acids or any appropriate combinations thereof.

The effervescent tablet includes between about 200 mg and about 800 mg of an edible binding excipient material. For example, the effervescent tablet may include between about 250 mg and about 600 mg of the binding excipient material. Suitable binding excipient materials include non-hygroscopic and low-hygroscopic ingredients and sugar alcohols. Mannitol is frequently used, since it has potential medicinal characteristics and lacks a strong taste. However, other sugar alcohols may be employed. The edible binding material ranges from 200 mg to 800 mg, preferably 250 mg to 600 mg.

In the pharmaceutical, supplement and food arts, when tablets, powders and granules are prepared for dilution or dissolution into water, it is common to include compounds known as disintegrants, which causes them to disintegrate and release their medicinal substances on contact with moisture. Exemplary disintegrants include but are not limited to starches and organic edible fiber. Due to the nature of its ingredients, the effervescent tablet tends to readily disintegrate and mix into solution with the water into which it has been mixed. Thus, in preferred embodiments, the effervescent tablet is free of such disintegrants.

Compounds known as lubricants are also frequently added to tablets, powders and granules, to provide free flowing of the granulates in manufacturing equipment and easy release of tablets from the molds and punches of the tablet presses. Exemplary lubricants include but are not limited to magnesium stearate, calcium stearate, stearic acid and sodium lauryl sulfate. Because lubrication excipients are hydrophobic and tend to produce insoluble residual suspensions in water, their use is very limited in the effervescent tablets. Therefore, in preferred embodiments, the effervescent tablet is substantially free of lubricants; however, either disintegrates or lubricants may be used.

As mentioned before, none of the known lubricant excipients are suitable for producing drinking water additive. However, the present invention provides a novel solution by using fumaric acid simultaneously as an active ingredient, as a flowing agent and as a lubricant when preparing the effervescent tablet of the preferred invention. The lubricating abilities of fumaric acid are achieved by reducing the particle size to 10-microns and by blending the resulting material in the tablet compounding powder.

As noted above, the effervescent material is provided to consumers. When packaged in a sealed container with a desiccant, such as is known in the art, the effervescent material is substantially non-degradable for at least 24 months. In some embodiments, when the effervescent material is sealed with a desiccant, the effervescent material is substantially non-degradable more than 24 months. For example, when provided in proper packaging, the effervescent material is substantially non-degradable for at least 48 months.

In another embodiment, a method of making the above described effervescent material for the generation of molecular hydrogen (H₂) in a quantity of water is provided. To make the effervescent material, quantities of the base metal Me, the dry organic acid and the edible binding material are provided and then mixed together. The lubricating ingredients are micronized to a size of between 5 microns and 10 microns.

To use, the effervescent table can simply be added to potable water. Bottled water, including clean tap water, reverse osmosis water, distilled water and all forms of mineral water are suitable waters with which the effervescent tablet can be used. The tablet can be added to any volume of water, preferably between 200 ml and 2 liters of water, and most preferably 500 ml of water. After adding the tablet, the solution should be allowed to rest, sit or percolate for at least five minutes before consumption. Preferably, the solution should be allowed to rest for at least 15 minutes, and more preferably 20 minutes. Allowing the solution to rest less than five minutes should not harm the drinker, though the solution will be less palatable than when the solution rests for a full five minutes.

It is to be understood that while certain forms of the present invention have been illustrated and described herein, it is not to be limited to the specific forms or arrangement of parts described and shown.

What is claimed and desired to be secured by Letters Patent is as follows:

1. An effervescent tablet, for the generation of molecular hydrogen in water, comprising:
a) a quantity of a base metal ranging from between about 5 mg and about 200 mg of the base metal;
b) a quantity of an edible non-hydroscopic dry organic acid ranging from between about 20 mg and about 400 mg of the organic acid, wherein the quantity of the organic acid corresponds with the quantity of the base metal, wherein stoichiometrically the limiting reactant is such that the proton concentration is lower than 1e⁻⁷M; and
c) a quantity of an edible binding excipient material ranging from between about 200 mg and about 800 mg of the binding excipient material; wherein
d) when diluted in a quantity of water to a mixture including between about 200 ml of water and about 2 liters of water, the effervescent tablet yields a hydrogen concentration between about 0.8 mM hydrogen and about 3 mM hydrogen and a final pH of the mixture of between about 8 and about 10.

2. The effervescent tablet according to claim 1, wherein:
a) the quantity of base metal is between 30 mg and 60 mg of the base metal.

3. The effervescent tablet according to claim 1, wherein:
a) the base metal is an active non-ionic metallic metal.

4. The effervescent tablet according to claim 1, wherein:
a) the base metal is selected from the group consisting of strontium, calcium, magnesium, aluminum, manganese, zinc, iron and mixtures thereof.

5. The effervescent tablet according to claim 1, wherein:
a) the edible dry organic acid is selected from the group consisting of an organic acid, an inorganic acid, a Lewis acid and mixtures thereof.

6. The effervescent tablet according to claim 1, wherein:
a) the quantity of the organic acid is from between about 60 mg and about 190 mg of the organic acid.

7. The effervescent tablet according to claim 1, wherein:
a) the quantity of the binding excipient material is from between about 250 mg and about 600 mg of the binding excipient material.

8. The effervescent tablet according to claim 1, wherein:
a) the quantity of water is about 500 ml of water.

9. The effervescent tablet according to claim 1, wherein:
a) the tablet is disintegrant-free.

10. The effervescent tablet according to claim 1, wherein:
a) the tablet is lubricant-free.

11. The effervescent tablet according to claim 1, wherein:
a) when packaged in the sealed container with a desiccant, the tablet is substantially non-degradable for a period of time of at least about 24 months.

12. The effervescent tablet according to claim 1, wherein:
a) the edible binding material is at least one of a non-hygroscopic, a low-hygroscopic ingredient and a sugar alcohol.

13. The effervescent tablet according to claim 1, wherein:
a) the edible binding material is mannitol.

14. The effervescent tablet according to claim 1, wherein:
a) the tablet includes from 200 mg to 800 mg of the edible binding material.

15. The effervescent tablet according to claim 1, wherein:
a) the tablet includes 250 mg to 600 mg of the edible binding material.

16. A method of making an effervescent tablet for the generation of molecular hydrogen in a quantity of water, comprising:
a) providing a quantity of a base metal, an edible dry organic acid and an edible binding material; and
b) providing a lubricating ingredient including fumaric acid having a particle size of between about 5 and about 10 microns.

17. The method according to claim 16, wherein:
a) the step of providing the lubricating ingredient includes providing fumaric acid having a particle size of about 5 microns.

18. The composition of claim 16 including the step of after constructing the tablet, adding the tablet to a bottle of water and immediately thereafter sealing the bottle, so as to retain released hydrogen in the water in the bottle.

19. An effervescent tablet for use with approximately 500 ml of water wherein the tablet comprises a base metal in the quantity of from about 5 mg to about 200 mg and an edible non-hydroscopic dry organic acid in a quantity from about 20 mg to about 400 mg.
